(19) United States
(12) Patent Application Publication
(10) Pub. No.: US 2016/0217260 A1
(43) Pub. Date: Jul. 28, 2016

(54) SYSTEM, METHOD AND COMPUTER PROGRAM PRODUCT FOR PATIENT TRIAGE

(71) Applicant: KONINKLIJKE PHILIPS N.V., EINDHOVEN (NL)

(72) Inventors: Ronaldus Maria Aarts, Geldrop (NL); Caifeng Shan, Eindhoven (NL); Radu Serban Jasinschi, Nuenen (NL)

(21) Appl. No.: 14/995,680

(22) Filed: Jan. 14, 2016

(30) Foreign Application Priority Data
Jan. 22, 2015 (EP) 15152063.2

(51) Int. Cl. G06F 19/00; A61B 90/00; G02B 27/01; H04N 5/247; G06K 9/00

(52) U.S. Cl. CPC G06F 19/327; H04N 5/247; G06K 9/00268; G06K 9/00295; G06K 9/00302; G06K 9/0061; G06K 9/00617; G06K 9/00778; G02B 27/0172; A61B 90/361; G02B 2027/0138; A61B 2090/502

(57) ABSTRACT

Disclosed is a system (100) for patient triage, comprising a plurality of cameras (110) for distribution across an area (10) such as a patient waiting room, scene of an accident or the like; a processor (120) for processing the respective image signals of said cameras and adapted to extract indicators for individual patients (20) in said area from said respective image signals, said indicators being indicative of the condition of said individual patients; and a prioritizing unit (130) adapted to prioritize the individual patients based on the indicators. Also disclosed are a patient triage method and a computer program product.

### CROSS REFERENCE TO RELATED APPLICATIONS

This claims the benefit of European Patent Application Number 15152063.2 filed Jan. 22, 2015, which is incorporated herein by reference.

### FIELD OF THE INVENTION

The present invention relates to a triage system for prioritizing patients for medical treatment.

The present invention further relates to a triage method for prioritizing patients for medical treatment.

The present invention yet further relates to a computer program product for implementing such a method.

### BACKGROUND OF THE INVENTION

Triage is the practice of prioritizing patients for medical treatments in scenarios where the number of patients seeking medical treatment exceeds the number of medical resources, e.g. medical practitioners such as consultants and/or nurses, such that a requirement exists to ensure that the patients in urgent need of medical attention are treated first.

There is an increased need for triage as there are increased pressures on many health systems, e.g. due to ageing populations and/or financial pressures on the health providers, which often prevents such providers to scale the available resources with increasing demand. Other well-known scenarios where triage is often necessary are during violent conflicts, large-scale accidents, terrorist attacks, epidemics or pandemics, to name but a few.

The associated increased pressure on the health providers further introduces the risk of human error, e.g. when implementing a triage procedure by a human, such as by a nurse or the like when inducting a patient into the pool of patients awaiting medical treatment. The pressure experienced by this person may for instance lead to an incomplete assessment of an arriving patient or a failure to observe changes to patients already inducted into the waiting process. This can cause patients needing urgent medical attention being overlooked or incorrectly prioritized, which can have grave consequences, e.g. death of the patient.

This has led to the introduction of (semi-)automated triage systems, an example of which is disclosed in US2013/0030825 A1. This example includes associating a patient with an identification bracelet and processing the patient using a patient evaluation device. The processing includes obtaining patient data with the patient evaluation device and dynamically determining a risk level associated with the patient based on the patient data obtained. The method also includes automatically prioritizing and scheduling the patient with a healthcare practitioner based on the risk level determined. The bracelet may include sensors for monitoring vital signs of the patient to dynamically update the risk level of the patient.

However, this system suffers from a few notable drawbacks. It still requires a degree of manual intervention in that the bracelet must be fitted to an incoming patient, which may be forgotten, e.g. in hectic circumstances. Moreover, a patient may lose the bracelet, may be unable to enter personal data into the system in the patient evaluation device or may enter incorrect data, all of which can prevent the system from operating in a satisfactory manner. There is therefore a need for a triage system that requires a reduced amount of human intervention.

### SUMMARY OF THE INVENTION

The present invention seeks to provide a system for patient triage that requires a limited amount of human intervention.

The present invention further seeks to provide a patient triage method requiring limited human intervention.

The present invention yet further seeks to provide a computer program product for implementing such a patient triage method.

According to an aspect, there is provided a system for patient triage, comprising a plurality of cameras for distribution across an area comprising patients awaiting treatment; a processor for processing the respective image signals of said cameras and adapted to extract indicators for individual patients in said area from said respective image signals, said indicators being indicative of the condition of said individual patients; and a prioritizing unit adapted to prioritize the patients based on their respective indicators.

The present invention is based on the insight that multiple cameras may be used to monitor multiple patients in an area such as a waiting room or the scene of an accident, with the image signals produced by these cameras containing information regarding the physical, i.e. medical, condition of these patients. This information may be extracted from these image signals using an appropriately configured signal processor, which extracted information is subsequently used to determine the physical condition of these patients and prioritize the patients accordingly using a prioritizing unit, which may be a separate unit or a unit incorporated by the image signal processor. In this manner, patients may be prioritized in a highly automated manner without having to use patient-attached monitoring devices, thus minimizing the amount of human intervention required, which reduces the risk of incorrect patient prioritization.

In an embodiment, said indicators include vital signs of individual patients, said vital signs including at least one of a breathing rate and a heart rate. Such vital signs indicators are particularly relevant to the physical condition of a patient and are therefore particularly suitable for use in a patient prioritization process.

The processor may alternatively or further be adapted to extract indicators from facial characteristics of an individual patient captured in said respective image signals, said facial characteristics including at least one of facial expressions and eye information.

In a particularly advantageous embodiment, the processor is further adapted to identify individual patients from the respective image signals. This may obviate the need for an initial (human intervention-based) patient registration process, in particular if the patients are already known to the system. This therefore has the potential to further reduce the amount of human intervention required in the patient triage.

The prioritizing unit advantageously may have access to a database of patient records and is adapted to prioritize the individual patients based on the indicators and the respective medical histories of the identified individual patients obtained from their patient records. By factoring in the medical history of a patient during patient triage, the quality of the patient prioritization decisions is potentially further improved.

The system may further comprise one or more sensors for capturing indicators of said conditions, wherein the one or more sensors optionally comprise at least one of an audio sensor and a temperature sensor. This facilitates the capturing of a wider variety of indicators of a patient's physical (medical) condition, such that this condition may be established at a higher level of confidence, thus further improving patient triage.

The audio sensor may be adapted to capture an indicator in the form of verbal responses of individual patients to one or more questions presented to the patient. Such questions may be automatically generated and the answers thereto automatically interpreted by the system. This may provide additional indicators of the condition of the patient, e.g. by determining if the patient is capable of answering questions in a coherent manner, thereby further increasing the data set of indicators on which the patient triage is based, which further improves the quality of the patient triage.

In an embodiment, the system comprises a head-mountable device including at least some of the cameras and/or one or more sensors. The head-mountable device may comprise an inward-facing image sensor for collecting an indicator in the form of eye information from an individual patient wearing the head-mountable device. Such eye information may be used to supplement the information extracted from the images captured by the cameras to further improve the quality of the patient triage.

The prioritizing unit may be adapted to prioritize said patients using a severity index-based decision model, for instance a binary model in which a severity index from a plurality of severity indices is assigned to a patient based on a series of yes/no-type decisions made based on the collected indicators for a particular patient, optionally supplemented by the available medical history of that patient.

To this end, the severity index-based decision model may implement a state machine responsive to said indicators and optionally further responsive to medical history information of said patients, said state machine comprising a plurality of severity states that can be populated through a plurality of transitions including a first transition for determining if a patient is in a critical condition; a second transition for determining if the behaviour of a non-critical patient is indicative of the patient requiring urgent attention; a third transition for determining the number of medical resources required to treat a patient not behaving in a manner indicative of the patient requiring urgent attention; and a fourth transition for assessing the condition of a patient based on monitored vital signs. Such a state machine-based model is particularly suitable to achieve high-quality patient triage.

In an embodiment, the system further comprises a further camera for collecting images of a patient in transit, wherein the processor is further adapted to extract one or more indicators of the condition of the patient in transit from the image signals provided by the further camera. This has the advantage that a patient in transit to the treatment waiting room may be assessed during transit such that a preliminary indication of the physical condition of the patient is already available upon arrival of the patient, such that the patient may be seen by a medical practitioner without undue delay for instance in case this preliminary indication signals that the patient is in a critical condition. This may reduce the delay between the patient arriving at the waiting room and the treatment of the patient, thereby improving the chances of recovery of the patient.

According to another aspect, there is provided a method for prioritizing patients awaiting treatment in an area, comprising monitoring said patients with a plurality of cameras distributed across said area; extracting indicators of the condition of individual patients from the image signals produced by said cameras; and prioritizing the patients based on their extracted respective indicators. This provides a highly automated patient triage method requiring minimal human intervention, which reduces the risk of incorrect patient prioritization. This method is preferably executed using an embodiment of the aforementioned system.

According to yet another aspect, there is provided a computer program product comprising a computer-readable medium carrying computer-readable program instructions for, when executed on a processor arrangement of a system for patient triage according to one or more of the aforementioned embodiments, implementing the aforementioned method of the present invention. Such a computer program product therefore facilitates the implementation of a patient triage requiring minimal human intervention.

### BRIEF DESCRIPTION OF THE DRAWINGS

Embodiments of the invention are described in more detail and by way of non-limiting examples with reference to the accompanying drawings, wherein:

FIG. 1 schematically depicts a patient triage system according to an example embodiment;

FIG. 2 depicts a flowchart of a patient triage method according to an example embodiment; and

FIG. 3 depicts an example embodiment of a severity index model that may be used by the patient triage system and/or the patient triage method according to one or more embodiments of the present invention.

### DETAILED DESCRIPTION OF THE EMBODIMENTS

It should be understood that the Figures are merely schematic and are not drawn to scale. It should also be understood that the same reference numerals are used throughout the Figures to indicate the same or similar parts.

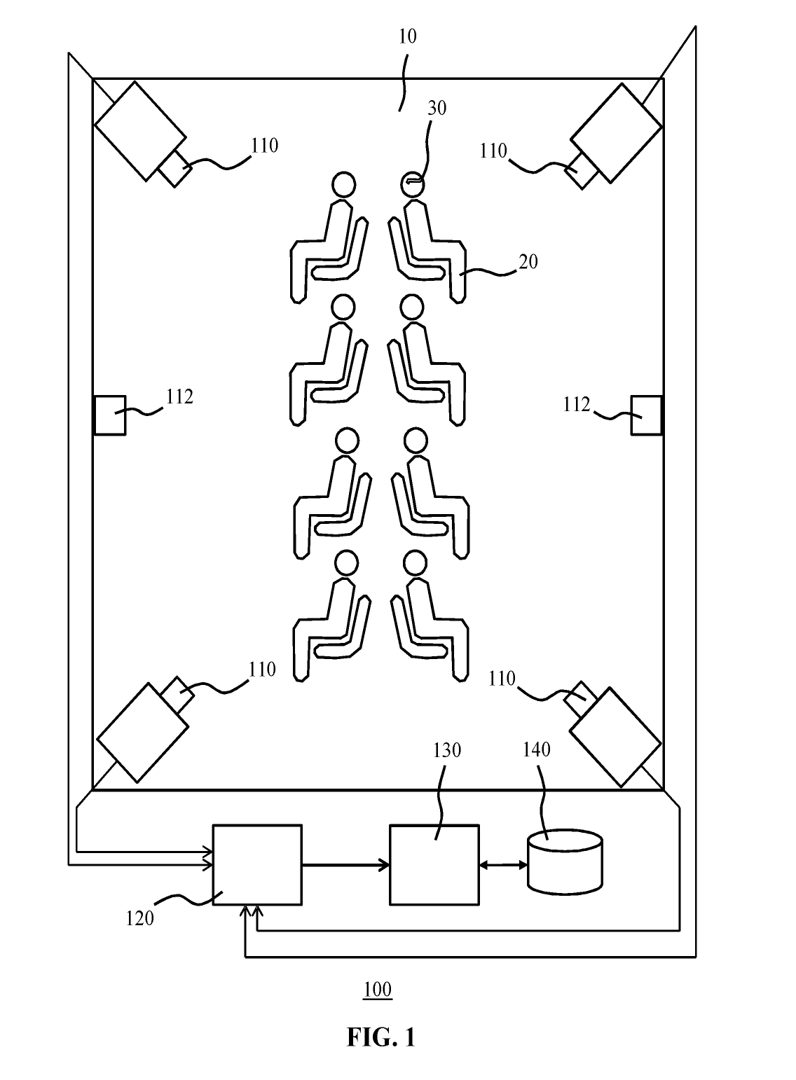

FIG. 1 schematically depicts a patient triage system 100 in accordance with an example embodiment of the present invention. The patient triage system 100 comprises a plurality of cameras 110 for distribution across a space or area 10 in which patients 20 await consultation and/or treatment by a medical practitioner, e.g. a waiting room of a medical facility such as a hospital emergency room or the like, the scene of an accident or other calamity, and so on. The cameras 110 are typically distributed across the space 10 such that each of the various locations in the space 10 in which a patient 20 may be located is monitored by at least one of the cameras 110. Such locations may be monitored by multiple cameras 110 in order to provide a level of redundancy in the patient monitoring, for instance to prevent loss of monitoring if the optical path between a camera 110 and the location is obscured for some reason. To this end, multiple cameras 110 monitoring the same location in the space 10 are preferably spaced apart such that when the optical path between one of the cameras and the location is obscured, the other camera(s) are still able to monitor that location.

In the case of a permanent location for holding patients, such as a waiting room, the cameras 110 may be fixed within the location. In more ad hoc spaces 10, e.g. the scene of an accident, the cameras 110 may be erected around the scene to monitor are patients, e.g. wounded, within the scene. Alternatively or additionally, if the scene includes fixed cameras, e.g. CCTV cameras, such fixed cameras may be used in addition or alternative to such erected (mobile) cameras 110. In an embodiment, at least some of the cameras 110 may be wearable cameras, e.g. form part of a wearable device such as a head-mountable device, e.g. smart glasses or the like, which may be worn by appropriate individuals, e.g. medical staff, to monitor the patients 20 in the space 10. This for instance is particularly advantageous in ad-hoc triage scenarios such as at the scene of major scale accidents, war zones and the like where permanently fixed or erectable mobile cameras may not be readily available or available in sufficient numbers to cover the entire scene.

The various cameras 110 are arranged to feed their captured images, e.g. a sequence of images captured at defined time intervals or a (near-)constant stream of images, e.g. a video stream, in the form of a plurality of image signals to a processor 120 for processing these image signals. This processor will be further referred to as a signal processor 120 although it should be understood that this is not intended to limit the functionality of this processor to signal processing only; it is equally feasible that the signal processor 120 is capable of performing other tasks as will be explained in more detail below.

The various cameras 110 may be arranged to provide the signal processor 120 with their respective image signals in any suitable manner, e.g. over a wired connection or over a wireless connection. Any suitable wireless communication protocol may be used for any of the wireless communication between a camera 110 and a signal processor 120, e.g., an infrared link, Zigbee, Bluetooth, a wireless local area network protocol such as in accordance with the IEEE 802.11 standards, a 2G, 3G or 4G telecommunication protocol, and so on.

Although not explicitly shown, wirelessly connected cameras 110 and the signal processor 120 may each comprise a suitable wireless communication interface for facilitating such wireless communication. As such wireless communication interfaces are well-known per se, this will not be explained in further detail for the sake of brevity only. It suffices to say that any suitable wireless communication interface may be used for this purpose. It should furthermore be understood that in some embodiments some of the cameras 110 are connected to the signal processor 120 in a wired fashion, whereas some other cameras 110 are connected to the signal processor 120 in a wireless fashion.

The signal processor 120 is typically arranged to extract indicators of the physical, i.e. medical, condition of a monitored patient from the image signals provided by the one or more cameras 110 monitoring this patient. In this manner, all patients 20 in the space 10 may be monitored by suitably placed cameras 110 such that the signal processor 120 can extract indicators of the physical condition of each of these patients from the respective image signals provided by the suitably placed cameras 110. Such indicators may include vital signs such as heart rate, breathing rate, blood pressure and so on, which can be derived from a sequence of images including a patient's face or chest for instance, which sequence of images may reveal subtle changes in the appearance of the patient such as changes in skin colour or chest movement, from which such vital signs may be derived.

For instance, heart rate may be measured by monitoring the skin area of a patient using a remote photo-plethysmography (PPG) technique, breathing rate may be measured by monitoring the subtle breathing motion in the belly or chest area of the patient and blood pressure may be measured using cameras if multiple skin regions of the patient (such as head and hand) are visible in the captured images, e.g. using a pulse transit time measurement technique as described in U.S. Pat. No. 8,838,209.

At this point it is noted that it is known per se that such vital signs can be derived from images captured by a camera. This is for instance disclosed in US 2014/303454 A1 and WO2013/186696 A1. The applicant for instance markets such a camera under the name Vital Signs Camera, a description of which can be found on the Internet at http://www.vitalsignscamera.com/. As extraction of such vital signs from an image signal is well-known per se, this will not be explained in further detail for the sake of brevity only. It is simply stated that any suitable extraction method may be employed by the signal processor 120. In addition to the aforementioned vital signs, further indicators that may be extracted from the image signals include but are not limited to: noticeable perspiration, facial expressions and/or body movements that may be indicative of distress or pain, changes in temperature in case the cameras 110 include thermal cameras, and so on.

In an embodiment, the system 100 further comprises a further camera (not shown) in wireless communication with the signal processor 120, which further camera is typically fitted in an ambulatory context, e.g. fitted in a vehicle such as an ambulance or to a bed on wheels, such that a patient 20 in transit to the space 10 may be monitored by the further camera, with the signal processor 120 arranged to extract indicators of the physical condition of the patient in transit from the image signals produced by the further camera.

In an embodiment, the further camera may be a camera of a mobile communication device such as a tablet or mobile phone. This for instance facilitates a scenario in which a person accompanying the patient in transit such as a relative, friend or medical support personnel member operates the further camera on the mobile communication device to provide the signal processor 120 with the aforementioned image signals. This for instance may require the mobile indication device to establish a communications link with a system 100 through an authentication or login procedure, e.g. a mobile communications-based or an Internet-based procedure, which may be used to forewarn the system 100 that a patient is on his or her way to the space 10, after which the further camera may be used to provide the image processor 120 with the image signals of the images captured at the patient in transit. The initial establishing of the communications link may include providing identification information of the patient in transit to further reduce the amount of human intervention required once the patient arrives at the space 10. However, as will be explained in further detail below, other identification techniques of the patients 20 are equally feasible.

The system 100 further comprises a prioritizing unit 130 for prioritizing the patients 20 based on the various indicators determined for each of these patients 20. An example embodiment of the operation of the prioritizing unit 130 will be explained in further detail below. The prioritizing unit 130 may be a separate unit, e.g. a separate processor or may form part of the signal processor 120. It should be understood that the system 100 may have any suitable processor arrangement for implementing the functionality of the signal processor 120 and the prioritizing unit 130.

The system 100 may optionally comprise one or more sensors 112 for monitoring some of the indicators of the physical condition of the patients 20, e.g. the patients 20 within the space 10 or in transit thereto. Such sensors 112 may be located in any suitable location, e.g. distributed across the space 10, and may be arranged to provide the sensor signals from which these indicators can be extracted to the signal processor 120 or to the prioritizing unit 130 for extraction of these indicators from the sensor signals. Alternatively, at least some of the sensors 112 may extract these indicators from the obtained sensor signals and directly forward the extracted indicators to the prioritizing unit 130. The sensor signals may be coupled to a particular patient 20 based on the location of the sensor 112 within the space 10. To this end, the sensors 112 may provide identification information or location information that allows the system 100 to associate the sensor signals with a particular patient 20, e.g. a particular patient 20 within the space 10 or in transit.

The one or more sensors 112 may communicate with the signal processor 120 and/or the prioritizing unit 130 in any suitable manner, e.g. using wired or wireless communication. Any suitable form of wireless communication such as previously explained for the communication between the cameras 110 and the signal processor 120 may be employed for this purpose.

In an embodiment, the one or more sensors 112 include an audio sensor such as a microphone or the like for capturing an indicator in the form of audible output of a patient, e.g. moaning, groaning or the like, the nature of which audible output may be interpreted to determine (at least in part) the physical condition of that patient.

Such an audio sensor may further be used to capture responses of the patient to one or more questions, which responses may be used in the assessment of the physical condition of the patient. For example, the system 100 may determine the response time to a question and/or the answer to the question, which answer for instance may be a statement of physical condition or may be used to derive an indicator of the physical condition of the patient, for instance by determining if the answer makes sense, which can be an indicator of the patient being in a disoriented state and/or suffering from a brain injury. Such patient interrogation may be performed in any suitable location, such as in a reception area of the space 10. The questions may be put to the patient by a human such as a duty nurse or may be generated by the system 100 using artificial speech routines. In such a scenario, the system 100 may further comprise one or more loudspeakers over which the questions can be put to the patient. The system 100 may interpret the answers of the patient to the questions in any suitable manner, such as by using voice recognition algorithms. As such speech and voice recognition algorithms are well-known per se, this will not be explained in further detail for the sake of brevity only. It suffices to say that any suitable algorithm may be used for this purpose.

In an embodiment, the one or more sensors 112 include a temperature sensor for sensing the body temperature of a patient 20 in transit or in the space 10, as body temperature is of course a useful indicator of the physical condition of a patient. Other suitable sensors will be apparent to the skilled person.

In an embodiment, at least some of the patients 20 may be provided with a head-mountable device 30 of the system 100, which head-mountable device 30 may include a camera 110 and/or at least one sensor 112, such that the physical condition of the patient 20 may be monitored when the patient 20 is on the move and out of reach of the stationary cameras 110 and/or sensors 112, e.g. when the patient 20 is visiting a restroom or leaves the space 10 for other reasons. The head-mountable device 30 equally may be provided to stationary patients 20 in the space 10, e.g. to provide supplementary indicators of the patient's physical condition. To this end, the head-mountable device 30 typically comprises one or more wireless communication interfaces for wirelessly communicating with the signal processor 120 and/or the prioritizing unit 130 of the system 100 using any of the aforementioned suitable wireless communication protocols. The head-mountable device 30 may take any suitable form, e.g. a wearable headband, hat, cap, glasses, and so on. Smart glasses are particularly preferred.

The head-mountable device 30 may include an inward facing image sensor for collecting an indicator in the form of eye information from an individual patient 20 wearing the head-mountable device 30. Such eye information, e.g. iris information, pupil dilation, and so on, may be used to generate a stand-alone indicator or to supplement the information from another source, e.g. a stationary camera 110 in the space 10, to extract an indicator from the combined information. As will be explained in further detail below, such eye information may also be used to (help) identify a patient.

In an embodiment, the prioritizing unit 130 has access to a patient database 140, which may form part of the system 100 or may be a separate database. The patient database 140 typically comprises the medical histories of patients previously treated in the medical facility or an affiliated medical facility sharing the patient database 140. In this embodiment, the prioritizing unit 130 may prioritize the patients 20 based on the indicators of the respective physical conditions of these patients as previously explained combined with medical history information of these patients. This for instance may be used to identify patients having known critical conditions that for some reason however do not exhibit any signs indicating that the patient may be in such a critical condition, wherein such patients with a medical history giving cause for concern may be prioritized over patients having a less critical medical history or no medical history at all.

The above embodiments of the system 100 may be arranged such that the indicators collected by the system 100 are periodically updated, e.g. by periodically repeating the capturing of such indicators to ensure that the assessment, i.e. prioritization, of the respective patients 20 in transit or in the space 10 is kept up-to-date. Any suitable update frequency may be employed, e.g. once every few minutes, once per minute, several times per minute, although it should be understood that other update frequencies are equally feasible.

It is furthermore noted that it is not necessary that the ultimate patient prioritization decision is made by the system 100. It is equally feasible that the system 100 provides the medical professionals with an assessment of the physical condition of each of the patients 20 such that the medical professionals can prioritize the patients 20 based on the respective assessments. In this embodiment the prioritizing unit 130 for instance may be adapted to prioritize the individual patients based on the indicators, wherein the prioritization comprises distributing the patients 20 within a severity index model having different levels of severity of condition, such that the medical professionals may select individual patients for treatment from the appropriate severity level, e.g. the highest severity level to which at least one patient is assigned by the prioritizing unit 130.

The system 100 may comprise any suitable user interface(s) such as a data input interface, e.g. a keyboard, track ball, mouse, touch screen, microphone and so on for allowing the input of data into the system, and/or a data output interface such as a display screen, loudspeaker or the like to provide a user with data output, such as the prioritization results produced by the prioritizing unit 130.

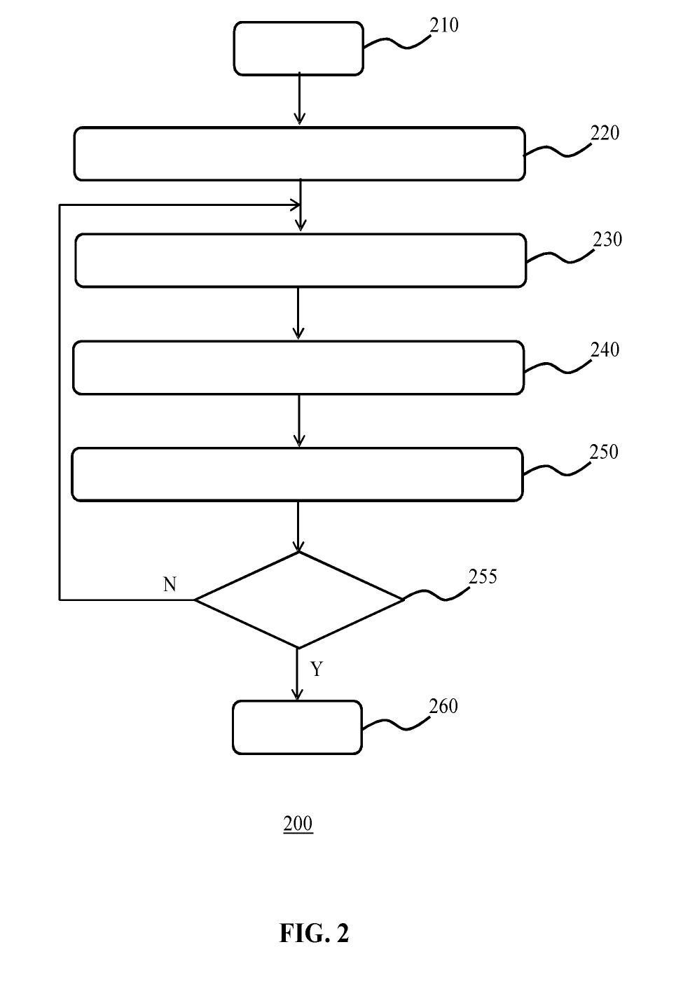

A patient triage method 200 implemented by the system 100 will now be explained in further detail with the aid of FIG. 2, which depicts a flow chart of an example embodiment of this method 200. The method 200 starts in step 210, e.g. by the arrival of a patient 20 in a medical facility housing the space 10 or by the collection of a patient 20 from a remote location for transfer to the medical facility, e.g. in case of an emergency. Alternatively, this step may be omitted in case of an ad-hoc triage event where patients are treated at the scene of the event as previously explained.

The method may subsequently proceed to optional step 220 in which the patient 20 is identified by the system 100. Such identification may be achieved automatically by the system 100 by way of face recognition or other suitable biometric identification of the patient 20. To this end, the signal processor 120 may employ one or more face recognition (or other biometric identification) algorithms that interpret the image signals provided by one or more cameras 110 (or by one or more further cameras monitoring one or more patients in transit) to identify the patients from these image signals. In an embodiment, this may involve retrieving a facial image of the patient from the patient database 140 and comparing the retrieved facial image with a facial image extracted from the image signals provided by camera(s) 110 to identify the patient. It should however be understood that any suitable face recognition technique may be employed by the system 100.

In case the patient is not recognized by the system 100 or in case the system 100 does not employ automatic patient identification through face recognition, the system 100 may capture a facial image of the patient to create a new patient record, which patient record may further be updated by duty staff dealing with the induction of the patient into the system 100, e.g. by filling in patient details such as patient name, address and age, medical history, and so on. In an embodiment, the newly created patient record may be stored in the patient database 140.

In an embodiment, such a patient record may be automatically updated by the system 100 by providing the patient with a series of questions, which questions may be provided to the patient using a suitable user interface such as a display or audio output device such as a loudspeaker, wherein the system 100 is adapted to capture the answers of the patient to these questions and populate the patient record in accordance with the captured answers. The answers may be captured in any suitable manner, e.g. using an audio input device such as a microphone, which audio input may be interpreted using voice recognition algorithms, using a user interface such as a keyboard or touch screen that allows the patient (or a companion) to provide the requested information, and so on. In this embodiment, a duty staff member will only be required to intervene if the patient is incapable of providing the requested information in an automated manner, for instance because the patient is an unfit physical condition to provide the requested information in such a manner.

Once the patient has been identified, the method 200 proceeds to step 230 in which the physical condition of the patient is monitored by capturing indicators of this condition, e.g. vital signs and supplementary indicators, using the (further) cameras 110 and optionally one or more sensors 112 as previously explained. The signals generated by the cameras 110 and optional sensors 112 are processed by the system 100, e.g. by the signal processor 120 and/or any other suitable processor (not shown) in step 240 to extract the indicators of the physical condition of the patients 20 from these signals, as also explained above.

The extracted indicators are processed by the prioritizing unit 130 in step 250 in order to prioritize the patients 20 in the space 10 or in transit thereto to determine the order in which the patients 20 should be seen by the medical staff or at least provide a classification of the patients 20 in terms of severity of their condition such that the medical staff can rely on this classification when selecting the next patient for treatment. To this end, the prioritizing unit 130 may implement a decision making model using the indicators as parameters, which decision making model may further take into account the medical history of the patients 20 in case such a medical history is available, for instance if the prioritizing unit 130 has access to the medical database 140 as previously mentioned. The decision making model may be a binary model comprising a number of states or levels of condition severity, wherein binary decisions, e.g. yes/no decisions, are made based on one or more of the indicators, which decisions determine if a patient to be prioritized should be assigned to a particular severity level.

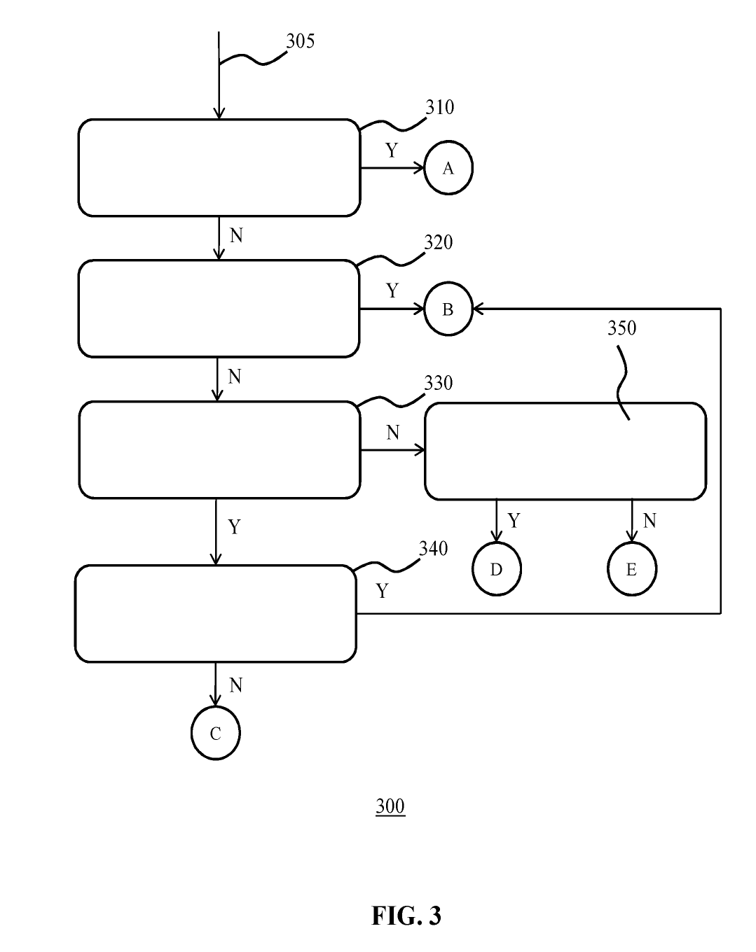

A non-limiting example of such a decision making model 300 is schematically depicted in FIG. 3. The decision making model 300 can be seen as a finite state machine comprising a number of decision-making transitions through which a number of states indicative of the severity of the condition of the patient may be populated. Such transitions may all emanate from the same initial state, e.g. an assessment state, and may implement a condictional decision tree with the condition severity states being terminal nodes of this decision tree. Such nodes may be arranged at different depths of the decision making tree, e.g. the transitions may define a sequence of IF THEN ELSE decisions, where a THEN branch may assign a patient to a condition severity state, i.e. may indicate a precondition for entering such a state having been met, whereas an ELSE branch may assign the patient to the next transition, e.g. assessment, i.e. may indicate a precondition for entering such a state not having been met. In FIG. 3, the decision making model comprises five of such severity-indicating states, which are labeled A, B, C, D and E respectively, with A indicating the most severe physical condition, i.e. indicating the patients in most urgent need of medical attention and E indicating the least severe medical condition.

The decision making model 300 comprises a symbolic input 305, which indicates the provision of the indicators and optionally the medical history for a patient under consideration. These inputs will be collectively referred to as the input parameters (of the decision making model 300). At least some of the input parameters are first assessed in decision making module or transition 310 to determine if the patient exhibits primary critical signs, e.g. is intubated, apneic, pulseless or unresponsive, e.g. by analysing the image data provided by the cameras 110 and optionally the sensors 112. For instance, whether a patient is intubated can be detected by analysing the acquired image/video through one of the cameras 110.

The vital signs of the patient, such as heart beat and respiration, can also be monitored with a camera 110 as previously explained. Based on this, the system can detect if the patient is apnoeic or pulseless. The responsiveness of the patient may further be measured by monitoring the interaction between the patient with the system or other people, e.g. by the detection of responses and/or response times to targeted questions as previously explained, from which the system can decide the responsiveness level of the patient.

Detected facial expressions may also be used as an indicator; in this case a set of template facial expressions may be employed by the system 100 to determine the patient's expression and map the determined facial expression to a known set of conditions, e.g., pain, numbness, unconsciousness. Also, a captured image of the eye and the retina may be used, for instance where a patient has been injured in a blast, car/motorcycle accident or the like, to determine a traumatic brain injury (TBI) score as a separate indicator.

If the patient exhibits one or more of these indicators as assessed in module 310, the prioritizing unit 130 may place the assessed patient in severity state A. If the assessed patient on the other hand does not clearly exhibit any of these indicators, the prioritizing unit 130 may decide that the patient does not qualify for the highest level of priority such that the patient assessment may be passed onto the next decision module or transition 320.

In this next module, the prioritizing unit 130 may determine the level of consciousness of the patient. It is noted that the patient must have some level of consciousness given that unconscious or unresponsive patients have been placed by the prioritizing unit 130 in severity state A. The level of consciousness may be an indicator of the risk that the patient may become critical. In the decision module 320, the prioritizing unit 130 for instance may use input parameters such as the responsiveness of the patient to certain requests, e.g. the ability of the patient to answer certain questions and/or visual indicators of such a level of consciousness. For instance, the patient may exhibit visible signs of concussion, stains of blood, which may be determined by the one or more cameras 110. The patient may further exhibit signs of being confused, lethargic or disoriented, which again may be visually identified from the position or movement of the head of the patient, e.g., head rocking, facial expressions such as frowning of the forehead or the shape of the mouth, the state of the patient's eyes, e.g., reddish, dilated pupils, and so on. The patient may further exhibit non-typical behaviour, e.g., exhibit unusually slow movement of the head, torso, arms and/or hands, which again may be detected by the signal processor 120 from the image signals provided by the one or more cameras 110, which in such a scenario preferably are video signals or sequence of still images taken at a high enough frequency to facilitate the detection of such movement.

The level of consciousness of a patient may further be determined using captured audio, e.g. using an audio sensor 112, for instance to detect anomalies in the speech of the patient, e.g., slurring, or to detect indications of severe pain or distress, e.g. a patient crying or moaning or providing a spoken indication of which part of the body hurts. In an embodiment, the system 100 may further or alternatively employ gesture recognition such that the system can recognize indications of for instance severe pain or distress by a patient gesturing. Such gestures for instance may include pointing to affected body parts. Another example of gesture is ‘grasping’ which may occur e.g. due to brain trauma or may be an indication of the patient being in a delirious state.

Based on the above indicators assessed in decision module or transition 320, the prioritizing unit 130 may decide that a patient should be placed in the second-highest severity state B, for instance if the patient is lethargic or confused or exhibits symptoms of being in severe pain or distress. If on the other hand the patient does not exhibit any of these symptoms, the prioritizing unit 130 may decide against placing the patient in severity state B and instead forward to decision making process to the next decision making module or transition 330.

In module 330, the prioritizing unit 130 may determine how many different resources are needed to treat the patient. This can be based on the pre-defined rules, which defines for each situation the resources are needed. To this end, the system 100 may have access to a medical resource information system such that the system 100, e.g. the prioritizing unit 130 can determine which resources are available at that point in time. Such resources may include different types of medical equipment for instance, such as a MRI scanner, CT scanner, X-ray apparatus, and so on.

The number of resources required by the patient may be used as an indication of the severity of the condition of the patient. For example, if the prioritizing unit 130 determines that the patient requires more than a predetermined number of resources, e.g. more than one resource, this is a likely indication of the patient being in a serious condition, in which case the prioritizing unit 130 may applied the next decision module 340 in which the vital signs of the patient such as heart rate, respiration rate and blood oxygen levels are determined and compared to benchmark values to determine if these vital signs are critical. If this is the case, the prioritizing unit 130 may place the patient in severity state B, otherwise, i.e. if the vital signs are not critical, the prioritizing unit 130 may place the patient in severity state C.

In an embodiment, the aforementioned benchmark values of the vital signs may be age-dependent and/or gender-dependent. In this embodiment, the prioritizing unit 130 may therefore assess the monitored vital signs as a function of the age and/or gender of the patient. The age and/or gender of the patient may be determined upon induction of the patient into the system 100, e.g. by asking the patient or his or her companion to specify the age and/or gender of the patient, by retrieval of the age and/or gender of the patient from the patient database 140 if the patient has a record in that database or by estimating the age and/or gender of the patient from the images of the patient captured by the one or more cameras 110.

If the prioritizing unit 130 determines in decision module 330 that the patient does not require more than a predetermined number of resources, this may be interpreted as the patient not being in a particularly serious condition, in which case the prioritizing unit 130 may determine in decision module or transition 350 if the patient requires the predetermined number of resources, e.g. a single resource, in which case the patient is placed in severity state D if the patient requires the predetermined number of resources, or is placed in severity state E if the patient requires less than a predetermined number of resources, e.g. no resources at all.

In this manner, the prioritizing unit 130 may categorise the patients 20 in different states or categories of severity, wherein medical staff may use these categorisations to determine which patients should be treated first. Alternatively, this determination may be made by the prioritizing unit 130 of the system 100 to further minimize the amount of human intervention required. At this point, it is noted that the decision making model 300 as explained above is merely an example of a suitable decision making model. Such decision making models are known per se and it should be understood that any suitable decision making model may be employed by the prioritizing unit 130.

Upon returning to FIG. 2, after the prioritizing unit 130 has prioritised patients 20 in step 250, the method may proceed to step 255 in which it is decided if the patient prioritization process should be repeated, e.g. updated, for instance to ensure that changes in the conditions of some of the patients 20 are captured by the system 100. If such updating is required, the method 200 will typically revert back to step 230 in which a fresh set of indicators of such conditions are captured by the system. Otherwise, the method 200 will terminate in step 260.

Aspects of the present invention may be embodied as a patient triage system 100 or a patient triage method 200. Aspects of the present invention may take the form of a computer program product embodied in one or more computer-readable medium(s) having computer readable program code embodied thereon. The code typically embodies computer-readable program instructions for, when executed on a processor arrangement of such a patient triage system 100, implementing the patient triage method 200.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Such a system, apparatus or device may be accessible over any suitable network connection; for instance, the system, apparatus or device may be accessible over a network for retrieval of the computer readable program code over the network. Such a network may for instance be the Internet, a mobile communications network or the like. More specific examples (a non-exhaustive list) of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present application, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out the methods of the present invention by execution on the processor 110 may be written in any combination of one or more programming languages, including an object oriented programming language such as Java, Smalltalk, C++ or the like and conventional procedural programming languages, such as the “C” programming language or similar programming languages. The program code may execute entirely on the processor 120 and/or the prioritizing unit 130 as a stand-alone software package, e.g. an app, or may be executed partly on the processor 120 and/or the prioritizing unit 130 and partly on a remote server. In the latter scenario, the remote server may be connected to the system 100 through any type of network, including a local area network (LAN) or a wide area network (WAN), or the connection may be made to an external computer, e.g. through the Internet using an Internet Service Provider. Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions to be executed in whole or in part on the processor 120 and/or the prioritizing unit 130 of the system 100, such that the instructions create means for implementing the functions/acts specified in the flowchart and/or block diagram block or blocks. These computer program instructions may also be stored in a computer-readable medium that can direct the system 100 to function in a particular manner.

The computer program instructions may be loaded onto the processor 120 and/or the prioritizing unit 130 to cause a series of operational steps to be performed on the processor 120 and/or the prioritizing unit 130, to produce a computer-implemented process such that the instructions which execute on the processor 120 and/or the prioritizing unit 130 provide processes for implementing the functions/acts specified in the flowchart and/or block diagram block or blocks. The computer program product may form part of the system 100, e.g. may be installed on the system 100.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements. In the device claim enumerating several means, several of these means can be embodied by one and the same item of hardware. The mere fact that certain measures are recited in mutually different dependent claims does not indicate that a combination of these measures cannot be used to advantage.

1. A system for patient triage, comprising:
a plurality of cameras for distribution across an area comprising patients awaiting treatment;
a processor for processing the respective image signals of said cameras and adapted to extract indicators for individual patients in said waiting area from said respective image signals, said indicators being indicative of the condition of said individual patients; and
a prioritizing unit adapted to prioritize the patients based on their respective indicators.

2. The system of claim 1, wherein said indicators include vital signs of individual patients, said vital signs including at least one of a breathing rate and a heart rate.

3. The system of claim 1, wherein the processor is adapted to extract indicators from facial characteristics of an individual patient captured in said respective image signals, said facial characteristics including at least one of facial expressions and eye information.

4. The system of claim 1, wherein the processor is further adapted to identify individual patients from the respective image signals.

5. The system of claim 4, wherein the prioritizing unit has access to a database of patient records and is adapted to prioritize the patients based on their respective indicators and the respective medical histories of the identified patients obtained from their patient records.

6. The system of claim 1, further comprising one or more sensors for capturing indicators of said conditions, wherein the one or more sensors optionally comprise at least one of an audio sensor and a temperature sensor.

7. The system of claim 6, wherein the audio sensor is adapted to capture an indicator in the form of verbal responses of individual patients to one or more questions presented to the patient.

8. The system of claim 5, further comprising a head-mountable device including at least some of the cameras and/or the one or more sensors.

9. The system of claim 8, wherein the head-mountable device comprises an inward-facing image sensor for collecting an indicator in the form of eye information from an individual patient wearing the head-mountable device.

10. The system of claim 1, wherein the prioritizing unit is adapted to prioritize said patients using a severity index-based decision model.

11. The system of claim 10, wherein the severity index-based decision model implements a state machine responsive to said indicators and optionally further responsive to medical history information of said patients, said state machine comprising a plurality of severity states that can be populated through a plurality of transitions including:
a first transition for determining if a patient is in a critical condition;
a second transition for determining if the behaviour of a non-critical patient is indicative of the patient requiring urgent attention;
a third transition for determining the number of medical resources required to treat a patient not behaving in a manner indicative of the patient requiring urgent attention; and
a fourth transition for assessing the condition of a patient based on monitored vital signs.

12. A system of claim 1, further comprising a further camera for collecting images of a patient in transit, wherein the processor is further adapted to extract one or more indicators of the condition of a patient in transit from the image signals provided by the further camera.

13. A method for prioritizing patients awaiting treatment in an area, comprising:
monitoring said patients with a plurality of cameras distributed across said area;
extracting indicators of the condition of individual patients from the image signals produced by said cameras; and
prioritizing the patients based on their extracted respective indicators.

14. The method of claim 13, wherein said monitoring, extracting and prioritizing steps are performed with the system of claim 1.

15. A computer program product comprising a computer-readable medium carrying computer-readable program instructions for, when executed on a processor arrangement of a system for patient triage, implementing the method of claim 13.
